(19) United States
(12) Patent Application Publication
(10) Pub. No.: US 2021/0375479 A1
(43) Pub. Date: Dec. 2, 2021

(54) METHOD AND APPARATUS FOR PROCESSING ELECTRONIC MEDICAL RECORD DATA, DEVICE AND MEDIUM

(71) Applicant: BEIJING BAIDU NETCOM SCIENCE AND TECHNOLOGY CO., LTD., Beijing (CN)

(72) Inventors: Quan YUAN, Beijing (CN); Jun CHEN, Beijing (CN); Chao LU, Beijing (CN); Haifeng HUANG, Beijing (CN)

(21) Appl. No.: 17/116,972

(22) Filed: Dec. 9, 2020

(30) Foreign Application Priority Data
May 29, 2020 (CN) 202010478482.7

(51) Int. Cl. G16H 50/70; G16H 10/60; G16H 70/60; G16H 50/20; G06N 3/04

(52) U.S. Cl. CPC G16H 50/70; G16H 10/60; G06N 3/04; G16H 50/20; G16H 70/60

(57) ABSTRACT

Embodiments of the present disclosure disclose a method and apparatus for processing electronic medical record data, a device and a medium. An embodiment of the method includes: acquiring symptom entity data in electronic medical record data; obtaining symptom entity representation data based on the symptom entity data and a symptom entity representation model pre-obtained by training; the symptom entity representation model including a graph convolutional neural network layer; and obtaining a disease prediction result corresponding to the electronic medical record data, based on the symptom entity representation data and a classification model pre-obtained by training.

### CROSS-REFERENCE TO RELATED APPLICATIONS

This application claims priority to Chinese Patent Application No. 202010478482.7, filed with the China National Intellectual Property Administration (CNIPA) on May 29, 2020, which is incorporated herein by reference in its entirety.

### TECHNICAL FIELD

Embodiments of the present disclosure relate to computer technology, specifically to artificial intelligence technology, and more specifically to a method and apparatus for processing electronic medical record data, a device and a medium.

### BACKGROUND

With the continuous development and improvement of electronic information technology, electronic medical record systems have been widely popularized and used in hospitals. During a patient's consultation, a doctor may complete the record of medical information through an electronic medical record system, including medical course records, examination and inspection results, medical orders, surgical records, nursing records, etc. Automatic disease diagnosis refers to automatically predict a diagnosis result based on the information recorded by the doctor in the electronic medical record.

The electronic medical record generally contains two kinds of important information, one part is natural text information, and the other part is symptom entity information. For the symptom entity information in the electronic medical record, the existing technology usually uses an entity vector or one-hot form for the representation, and the accuracy is low, so that the accuracy of a diagnosis result predicted based on the symptom entity information is also low.

### SUMMARY

Embodiments of the present disclosure disclose a method and apparatus for processing electronic medical record data, a device and a medium, to improve the accuracy of disease prediction based on symptom entity information.

In a first aspect, some embodiments of the present disclosure provide a method for processing electronic medical record data, the method includes:

acquiring symptom entity data in the electronic medical record data;

obtaining symptom entity representation data based on the symptom entity data and a symptom entity representation model pre-obtained by training, the symptom entity representation model comprising a graph convolutional neural network layer; and

obtaining a disease prediction result corresponding to the electronic medical record data, based on the symptom entity representation data and a classification model pre-obtained by training.

In a second aspect, some embodiments of the present disclosure provide an apparatus for processing electronic medical record data, the apparatus includes:

a symptom entity data acquisition module, configured to acquire symptom entity data in the electronic medical record data;

a representation data acquisition module, configured to obtain symptom entity representation data based on the symptom entity data and a symptom entity representation model pre-obtained by training, the symptom entity representation model comprising a graph convolutional neural network layer; and

a disease prediction result acquisition module, configured to obtain a disease prediction result corresponding to the electronic medical record data, based on the symptom entity representation data and a classification model pre-obtained by training.

In a third aspect, some embodiments of the present disclosure provide an electronic device, the electronic device includes:

at least one processor; and

a memory, communicatively connected to the at least one processor; where,

the memory, storing instructions executable by the at least one processor, the instructions, when executed by the at least one processor, cause the at least one processor to perform the method for processing electronic medical record data according to any one of embodiments of the present disclosure.

In a fourth aspect, some embodiments of the present disclosure provide a non-transitory computer readable storage medium, storing computer instructions, the computer instructions, being used to cause a computer to perform the method for processing electronic medical record data according to any one of embodiments of the present disclosure.

According to the technical solutions of embodiments of the present disclosure, the symptom entity representation data are acquired based on the acquired symptom entity data and the symptom entity representation model pre-obtained by training, the symptom entity representation model including a graph convolutional neural network layer, and then the disease prediction result corresponding to the electronic medical record data is obtained based on the symptom entity representation data and the classification model obtained by pre-training. Since the symptom entity representation model pre-obtained by training includes the graph convolutional neural network layer, the output symptom entity representation data have high accuracy, so that the accuracy of the finally obtained disease prediction result corresponding to the electronic medical record data is also high.

### BRIEF DESCRIPTION OF THE DRAWINGS

The accompanying drawings are used to better understand the present solution and do not constitute a limitation to the present disclosure. In which:

FIG. 1A is a flowchart of a method for processing electronic medical record data disclosed according to Embodiment 1 of the present disclosure;

FIG. 1B is a schematic structural diagram of a symptom entity representation model disclosed according to Embodiment 1 of the present disclosure;

FIG. 1C is a schematic diagram of a medical knowledge graph disclosed according to Embodiment 1 of the present disclosure;

FIG. 2 is a schematic structural diagram of a symptom entity representation model disclosed according to Embodiment 2 of the present disclosure;

FIG. 3A is a flowchart of another method for processing electronic medical record data disclosed according to Embodiment 3 of the present disclosure;

FIG. 3B is a schematic diagram of a disease prediction disclosed according to Embodiment 3 of the present disclosure;

FIG. 4 is a schematic structural diagram of an apparatus for processing electronic medical record data disclosed according to Embodiment 4 of the present disclosure; and

FIG. 5 is a block diagram of an electronic device disclosed according to an embodiment of the present disclosure.

### DETAILED DESCRIPTION OF EMBODIMENTS

Example embodiments of the present disclosure are described below with reference to the accompanying drawings, which include various details of embodiments of the present disclosure to facilitate understanding, and should be considered merely as examples. Therefore, those of ordinary skill in the art should recognize that various changes and modifications can be made to the embodiments described herein without departing from the scope and spirit of the present disclosure. Likewise, for clarity and conciseness, descriptions of well-known functions and structures are omitted in the following description.

Automatic disease diagnosis is a core component of the clinical assistance system, which is used to provide a powerful assistance to the doctor's diagnosis. Fast and accurate automatic diagnosis results may greatly improve the efficiency of doctors' diagnosis, and significantly reduce the rate of misdiagnosis and missed diagnosis caused by the short of practitioners having professional capabilities.

The existing automatic disease diagnosis is mostly based on information in the electronic medical record. In the research and development phase, the applicant notes: 1) In the actual diagnosis process, the relationship between symptom entity information and a diagnosis result is very complicated, a disease may cause a variety of different symptoms, while a symptom may also be caused by many different kinds of diseases. This allows that the more accurate result of the disease diagnosis could be deduced when the symptom representation fuses as much disease information associated with the symptom as possible. 2) Due to the different writing habits of doctors in different hospitals, there may be differences in the expressions of the symptom entity information parsed from the electronic medical records, such as “brain hemorrhage” and “cerebral hemorrhage”, however in the existing technology they may be treated as two different symptom entities separately. As a result, this entity cannot be accurately and effectively learned and represented. Some symptom entities may have subtle differences in orientation or the like, but they actually express the same meaning, such as “formation of softening of the left basal ganglia” and “formation of softening of the right basal ganglia”, however they may also be regarded as different entities in the existing technology. As a result, this type of entity cannot be accurately and effectively learned and expressed. 3) For high-frequency symptom entities, such as “fever”, in the electronic medical records, it may have a very good expression effect. However, for low-frequency entities such as “eyelid hyperplastic macula”, electronic medical records corresponding thereto are relatively few, so it may be difficult to obtain a good expression.

Therefore, a method to improve the accuracy of expressing symptom information in electronic medical records is demanded, so as to make a final disease prediction result more accurate.

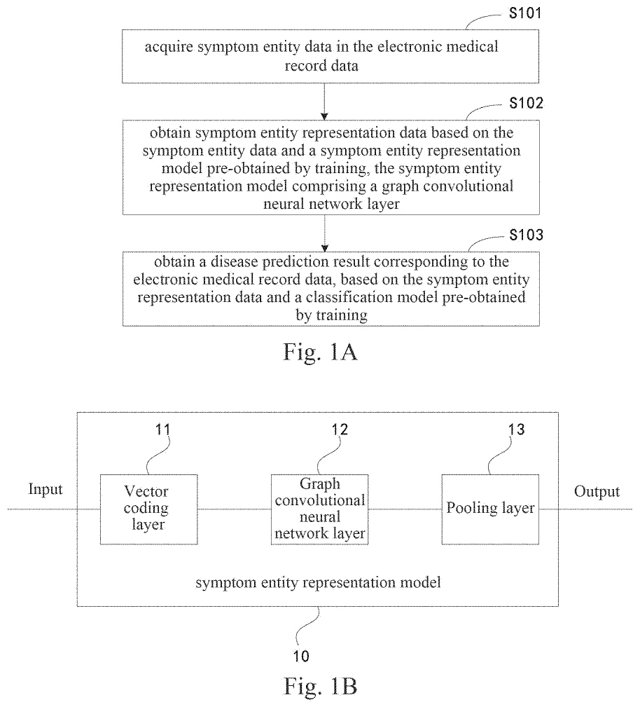

FIG. 1A is a flowchart of a method for processing electronic medical record data disclosed according to Embodiment 1 of the present disclosure. The present embodiment may be applied to the situation of automatically performing disease prediction based on electronic medical record data. The method in the present embodiment may be performed by an apparatus for processing electronic medical record data, which may be implemented by software and/or hardware, and may be integrated on any electronic device having computing capability, such as a server or a terminal device.

As shown in FIG. 1A, a method for processing electronic medical record data disclosed in the present Embodiment 1 may include:

S101, acquiring symptom entity data in electronic medical record data.

The symptom entity data are manually recorded into electronic medical records by doctors, or are automatically generated into electronic medical records by including natural language understanding technology to analyze patient oral content. The symptom entity data include but are not limited to patients' symptoms or abnormal signs, such as “cough”, “fever”, “sore throat”, “dyspnea”, “hoarse voice” and “wheezing”.

Specifically, the electronic medical record of a target patient is retrieved from an electronic medical record system, and symptom entity data are acquired from the electronic medical record. The acquisition method includes but is not limited to: 1) establishing a medical element partition in the electronic medical record in advance, the medical element partition is used to record symptom information of the patient, so that the symptom entity data may be directly extracted from the medical element partition of the electronic medical record. 2) extracting words related to “symptom” from the electronic medical record as the symptom entity data by using an existing domain-related word extraction algorithm. The acquired symptom entity data may be one piece of data or a plurality of pieces of data, and each piece of symptom entity data corresponds to a symptom or abnormal sign.

Alternatively, after acquiring the symptom entity data in electronic medical record data, the method further includes: storing the symptom entity data and patient information corresponding to the electronic medical record associatively into a database. By storing the symptom entity data and the patient information associatively into the database, it is possible to confirm relevant patient information more quickly when the symptom entity data are subsequently data backtracked.

By acquiring symptom entity data in electronic medical record data, data extraction of the symptom entity data is realized, which lays a data foundation for subsequent obtaining of symptom entity representation data based on the symptom entity data.

S102, obtaining symptom entity representation data based on the symptom entity data and a symptom entity representation model pre-obtained by training, where the symptom entity representation model includes a graph convolutional neural network (GCN) layer.

The symptom entity representation data are vectorized representation of the symptom entity data, and disease prediction may be realized based on the symptom entity representation data.

Specifically, the symptom entity data are input into the symptom entity representation model pre-obtained by training, and the output is the symptom entity representation data corresponding to the symptom entity data. The symptom entity representation model is provided with a graph convolutional neural network layer, for converting the symptom entity data into symptom entity representation data fused with graph structure information based on a pre-established medical knowledge graph. The medical knowledge graph includes disease entity nodes and symptom entity nodes, and there are a connection relationships between disease entity nodes and between the disease entity nodes and the symptom entity nodes.

Alternatively, FIG. 1B is a schematic structural diagram of a symptom entity representation model disclosed according to Embodiment 1 of the present disclosure, where the symptom entity representation model 10 includes: a vector coding layer 11, a graph convolutional neural network layer 12, and a pooling layer 13;

the vector coding layer 11 is used to encode the symptom entity data to obtain a symptom encoding vector corresponding to the symptom entity data.

Since the current processing device, such as computers, cannot process text content such as English or Chinese characters, it is necessary to convert the symptom entity data into a numerical form that the processing device can understand.

Specifically, after the symptom entity representation model 10 acquires the input symptom entity data, it transmits the symptom entity data to the vector coding layer 11. The vector coding layer 11 encodes the symptom entity data according to a preset encoding method, to obtain the symptom encoding vector corresponding to the symptom entity data. Here, the preset encoding method includes but is not limited to NNLM (neural network language model), word2vec, glove, ELMo, etc.

The graph convolutional neural network layer 12 is used to obtain symptom vectorized representation data fused with graph structure information based on the symptom encoding vector.

Specifically, the vector coding layer 11 transmits the output symptom encoding vector to the graph convolutional neural network layer 12. Based on the connection relationship between the disease entity nodes in the medical knowledge graph and the connection relationship between the disease entity nodes and the symptom entity nodes, the graph convolutional neural network layer 12 calculates and obtains the symptom vectorized representation data fused with graph structure information.

The pooling layer 13 is used to perform pooling processing on the symptom vectorized representation data to obtain the symptom entity representation data.

The function of the pooling processing is to reduce the amount data of the symptom vectorized representation data, and to reduce the overfitting of the symptom vectorized representation data.

Specifically, the graph convolutional neural network layer 12 transmits the output symptom vectorized representation data to the pooling layer 13, and the pooling layer 13 performs pooling processing on the symptom vectorized representation data according to a preset pooling method, to obtain the symptom entity representation data, where the preset pooling method includes a mean pooling processing method.

By setting a vector coding layer in the symptom entity representation model, the symptom entity data that cannot be recognized by a processing device are converted into the symptom encoding vector corresponding to the symptom entity data that recognizable by the device; by setting a graph convolutional neural network layer in the symptom entity representation model, the symptom vectorized representation data used to express the symptom entity data are fused with graph structure information of an associated disease, so that the accuracy of the symptom vectorized representation data is higher; and by setting a pooling layer in the symptom entity representation model, the resulting symptom entity representation data have a smaller data amount and the overfitting is avoid.

S103, obtaining a disease prediction result corresponding to the electronic medical record data, based on the symptom entity representation data and a classification model pre-obtained by training.

The classification model is used to determine, based on the symptom entity representation data, a disease prediction result corresponding to the electronic medical record data to which the symptom entity representation data belong. Training data for training the classification model may be acquired from a large number of high-quality electronic medical records in medical institutions with outstanding professional capabilities, such as the top class hospitals.

Specifically, the symptom entity representation data are input into the trained classification model, and the disease prediction result corresponding to the electronic medical record data to which the symptom entity representation data belong is output.

The disease prediction result corresponding to the electronic medical record data is obtained based on the symptom entity representation data and the classification model obtained by pre-training, and the effect of disease prediction for the patient based on the patient's electronic medical record data is realized.

According to the technical solution of the present embodiment, the symptom entity representation data is obtained based on the acquired symptom entity data and the symptom entity representation model pre-obtained by training, where the symptom entity representation model including the graph convolutional neural network layer, then, based on the symptom entity representation data and the classification model pre-obtained by training, the disease prediction result corresponding to the electronic medical record data is obtained. Since the symptom entity representation model pre-obtained by training includes the graph convolutional neural network layer, the symptom vectorized representation data used to express the symptom entity data are fused with the graph structure information of the associated disease, so that the accuracy of the symptom vectorized representation data is higher, and finally the accuracy of the obtained disease prediction result corresponding to the electronic medical record data is also high.

On the basis of the foregoing embodiment, before S101, the method further includes: constructing a medical knowledge graph.

The medical knowledge graph includes at least one disease entity node and at least one symptom entity node.

Specifically, a disease entity node represents a disease entity, such as “tracheitis”, “laryngotracheitis”, or “bronchitis” and “wheezing bronchitis”; a symptom entity node represents a symptom entity, such as “dyspnea”, “hoarse voice”, “wheezing”, “sputum expectoration” and “fever”.

There is a connection relationship between two disease entity nodes having a hyponymy relationship in the disease entity nodes.

For example, the disease entity node “fracture” is the hypernym of the disease entity node “humeral fracture”, so there is a connection relationship between the disease entity node “fracture” and the disease entity node “humeral fracture”, that is, the disease entity node “humeral fracture” belongs to one type of the disease entity node “fracture”. A certain disease entity node in the medical knowledge graph may have a plurality of hypernym disease entity nodes, or there may be a plurality of hyponym disease entity nodes.

For any disease entity node and any symptom entity node, if a disease corresponding to the disease entity node causes a symptom corresponding to the symptom entity node to occur, then there is a connection relationship between the disease entity node and the symptom entity node.

For example, the disease corresponding to the disease entity node “tracheitis” would cause symptoms corresponding to the symptom entity nodes “dyspnea” and “fever” to occur, then the disease entity node “tracheitis” has a connection relationship with the symptom entity nodes “dyspnea” and “fever”.

The connection relationships among the disease entity node, the symptom entity node, the disease entity node, and the connection relationships between the disease entity nodes and the symptom entity nodes in the medical knowledge graph of the present embodiment are mined from a large number of real desensitization medical records based on a statistical method. In the medical knowledge graph, the connection relationship between disease entity nodes has no weight, while the connection relationship between disease entity nodes and symptom entity nodes has a weight. This weight is obtained based on the frequency of the occurrence of the disease entity node, the greater the frequency, the greater the weight. Alternatively, since the connection relationship between disease entity nodes and symptom entity nodes has a long tail characteristic, and the connection relationship with a low weight is generally generated due to noise data, an overall effect will be affected if this part of low-weight edges are introduced into the calculation process. So the connection relationship associated with each symptom entity node is truncated, and only the connection relationship corresponding to a score in the Top-k range is retained. Preferably, k is set to 5, that is, each symptom entity node forms connection relationships with at most 5 disease entity nodes.

FIG. 1C is a schematic diagram of a medical knowledge graph disclosed according to Embodiment 1 of the present disclosure, including the disease entity nodes “tracheitis”, “laryngotracheitis”, “bronchitis” and “wheezing bronchitis”, the symptom entity nodes “dyspnea”, “hoarse voice”, “wheezing”, “sputum expectoration” and “fever”; the disease entity node “tracheitis” has a connection relationship with the disease entity nodes “laryngotracheitis” and “bronchitis” respectively, and the disease entity node “bronchitis” has a connection relationship with the disease entity node “wheezing bronchitis”; the symptom entity node “dyspnea” has a connection relationship with the disease entity nodes “tracheitis” and “laryngotracheitis” respectively, the symptom entity node “hoarse voice” has a connection relationship with the disease entity node “laryngotracheitis”, the disease entity node “wheezing” has a connection relationship with the disease entity node “wheezing bronchitis”, the disease entity node “sputum expectoration” has a connection relationship with the disease entity nodes “wheezing bronchitis” and “bronchitis” respectively, and the symptom entity node “fever” has a connection relationship with the disease entity nodes “tracheitis” and “bronchitis” respectively.

By constructing the medical knowledge graph, and constructing the connection relationship between the disease entity nodes and the connection relationship between the disease entity node and the symptom entity node in the medical knowledge graph, it lays the foundation for the subsequent the graph convolutional neural network to generate the symptom vectorized representation data fused with graph structure information based on the medical knowledge graph.

Correspondingly, the graph convolutional neural network layer is used to:

obtain the symptom vectorized representation data fused with graph structure information based on the medical knowledge graph and the symptom encoding vector.

Specifically, the graph convolutional neural network layer obtains the symptom vectorized representation data fused with graph structure information, based on the symptom encoding vector transmitted from the encoding layer, the connection relationships between the disease entity nodes, and the connection relationships between the disease entity nodes and the symptom entity nodes in the medical knowledge graph.

The symptom vectorized representation data fused with graph structure information are obtained based on the medical knowledge graph and the symptom encoding vector, so that the accuracy of the symptom vectorized representation data is higher.

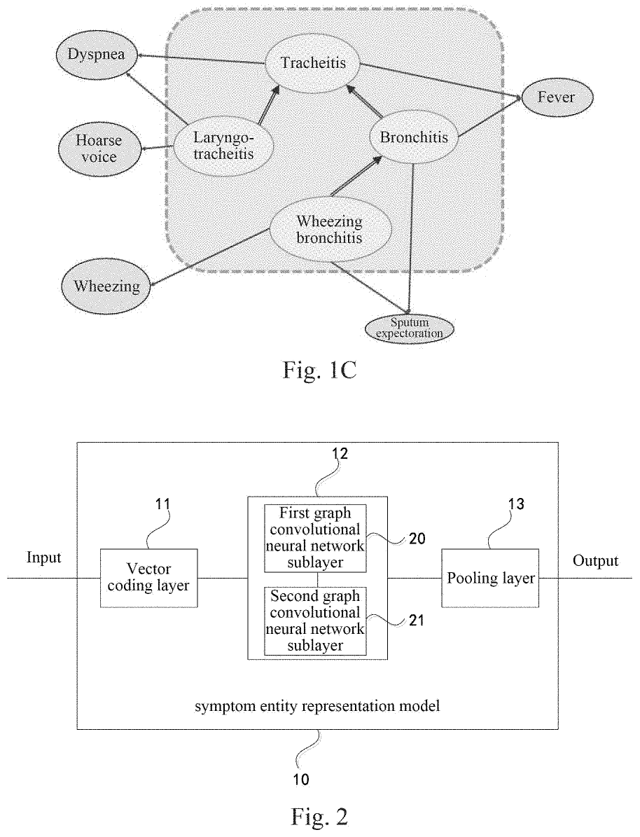

FIG. 2 is a schematic structural diagram of a symptom entity representation model disclosed according to Embodiment 2 of the present disclosure. The model further optimizes and expands the symptom entity representation model of FIG. 1B in the above Embodiment 1, and may be combined with the various alternative embodiments described above. As shown in FIG. 2, the symptom entity representation model 10 may include:

a vector coding layer 11, a graph convolutional neural network layer 12, and a pooling layer 13.

The graph convolutional neural network layer 12 includes a first graph convolutional neural network sublayer 20 and a second graph convolutional neural network sublayer 21.

the first graph convolutional neural network sub-layer 20 is used to obtain disease vectorized representation data fused with graph structure information, based on the medical knowledge graph and a disease encoding vector of a target disease entity node, the target disease entity node having a connection relationship with a target symptom entity node corresponding to the symptom entity data.

Specifically, the vector coding layer 11 determines, from the medical knowledge graph, a target disease entity node that has a connection relationship with the target symptom entity node corresponding to the symptom entity data, and encodes the target disease entity node to obtain the disease encoding vector corresponding to the target disease entity node, and finally transmit the symptom encoding vector and the disease encoding vector jointly to the graph convolutional neural network layer 12. The first graph convolutional neural network sublayer 20 in the graph convolutional neural network layer 12 acquires the disease encoding vector transmitted from the encoding layer 11, and in combination with the connection relationship between the disease entity nodes in the medical knowledge graph, to obtain the disease vectorized representation data fused with graph structure information.

Alternatively, the disease vectorized representation data fused with graph structure information are obtained according to the formula as follows:

$${\hat{D}}_{i} = ReLU ( {W}_{1} {D}_{i} + {\sum }_{u ∈ {N}_{p} ( i )} \frac{{W}_{2} {D}_{u}}{\vert {N}_{p} ( i ) \vert} + {\sum }_{v ∈ {N}_{c} ( i )} \frac{{W}_{3} {D}_{v}}{\vert {N}_{c} ( i ) \vert} + {B}_{1} )$$

ReLU represents an activation function, that is, the above formula may cause the sparsity of the model network and alleviate an overfitting problem; W₁, W₂, W₃, and B₁ respectively represent model parameters to be trained, values of W₁, W₂, W₃, and B₁ may be determined through model training, W₁, and W₃ are matrices of m*m-dimensions, B₁ is an vector of m-dimensions; Nₚ(i) represents a parent node set corresponding to the target disease entity node, for example, the disease entity node “bronchitis” has a connection relationship with the disease entity node “wheezing bronchitis”, the disease entity node “bronchitis” is an hypernym representation of the disease entity node “wheezing bronchitis”, then the disease entity node “bronchitis” is a parent node of the disease entity node “wheezing bronchitis”; N_{c}(i) represents a child node set corresponding to the target disease entity node, for example, the disease entity node “bronchitis” and the disease entity node “wheezing bronchitis” have a connection relationship, the disease entity node “wheezing bronchitis” is a hyponym representation of the disease entity node “bronchitis”, then the disease entity node “wheezing bronchitis” is a child node of the disease entity node “bronchitis”; \hat{D}ᵢ represents the disease vectorized representation data, Dᵢ represents the disease encoding vector; Dᵥ represents an encoding vector of a child node of the target disease entity node; Dᵤ represents an encoding vector of a parent node of the target disease entity node; |Nₚ(i)| represents the number of elements in the parent node set corresponding to the target disease entity node; and |N_{c}(i)| represents the number of elements in the child node set corresponding to the target disease entity node.

The effect of calculating the disease vectorized representation data fused with graph structure information can be achieved through the above formula.

The second graph convolutional neural network sublayer 21 is used to obtain the symptom vectorized representation data fused with graph structure information based on the medical knowledge graph, the symptom encoding vector, and the disease vectorized representation data.

Specifically, the first graph convolutional neural network sublayer 20 transmits the obtained disease vectorized representation data to the second graph convolutional neural network sublayer 21, and the second graph convolutional neural network sublayer 21 obtains the symptom vectorized representation data fused with graph structure information based on the symptom encoding vector acquired from the encoding layer 11 and the disease vectorized representation data acquired from the first graph convolutional neural network sublayer 20, in combination with the connection relationships between the disease entity nodes and the symptom entity nodes in the medical knowledge graph.

Alternatively, the symptom vectorized representation data fused with graph structure information are obtained according to the formula as follows:

$${\hat{F}}_{j} = ReLU ( {W}_{4} {F}_{j} + \frac{1}{\vert {N}_{g} ( j ) \vert} {\sum }_{i ∈ {N}_{g} ( j )} {A}_{i , j} {W}_{5} {\hat{D}}_{i} + {B}_{2} )$$

ReLU represents an activation function, W₄, W₅ and B₂ respectively represent model parameters to be trained, values of W₄, W₅ and B₂ may be determined through model training, W₄ and W₅ are are matrices of m*m-dimensions, B₂ is a vector of m-dimensions, N_{g}(j) represents a set of target disease entity nodes, that is, a set of disease entity nodes that have connection relationships with the target symptom entity node corresponding to the symptom entity data; A_{i,j} represents the weight of a connection relationship between a target symptom entity node and a target disease entity node; \hat{F}ⱼ represents the symptom vectorized representation data, Fⱼ represents the symptom encoding vector; and |N_{g}(j)| represents the number of elements in the target disease entity node set.

The effect of calculating the symptom vectorized representation data fused with graph structure information can be achieved through the above formula.

Alternatively, the weight A_{i,j} of a connection relationship between the target symptom entity node and the target disease entity node is determined according to the formula as follows:

$${A}_{i , j} = n {〈 {f}_{j} \vert {d}_{i} 〉}^{*} log \frac{N}{1 + n ( {d}_{i} )}$$

n [symbol] fⱼ|dᵢ [symbol] represents a frequency of the target symptom entity node presenting in medical records with the target disease entity node as the main diagnosis, that is, the number of times of that the target symptom entity node shows up in the medical records which are with the target disease entity node as the main diagnosis per unit time; n(dᵢ) represents the total number of the medical records which are with the target disease entity node as the main diagnosis; and N represents the total number of medical records used.

The effect of determining the weight of the connection relationship between the target symptom entity node and the target disease entity node is realized through the above formula.

In the present embodiment, by setting the graph convolutional neural network layer in the symptom entity representation model to include the first graph convolutional neural network sublayer and the second graph convolutional neural network sublayer, the first graph convolutional neural network sublayer is used to obtain the disease vectorized representation data fused with graph structure information based on the medical knowledge graph and the disease encoding vector; the second convolutional neural network sublayer is used to obtain the symptom vectorized representation data fused with graph structure information based on the medical knowledge graph, the symptom encoding vector and the disease vectorized representation data, so that the graph convolutional neural network can parse important medical knowledge graph structural features, improving the accuracy of the finally obtained symptom vectorized representation data, and the complexity of calculation and the computational time overhead can be effectively reduced.

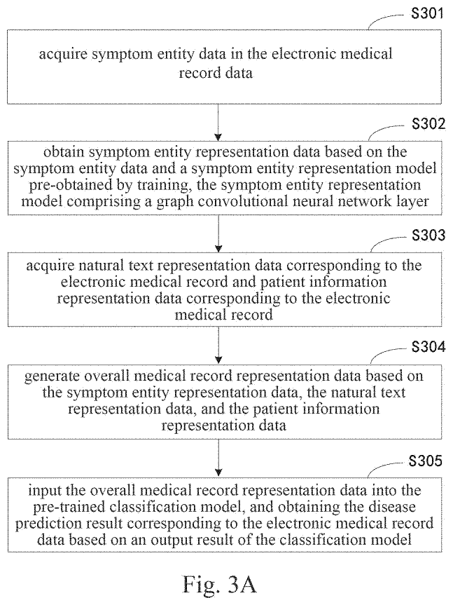

FIG. 3A is a flowchart of another method for processing electronic medical record data disclosed according to Embodiment 3 of the present disclosure, which further optimizes and expands the above technical solution, and may be combined with the above various alternative embodiments. As shown in FIG. 3A, the method may include:

S301, acquiring symptom entity data in electronic medical record data.

S302, obtaining symptom entity representation data based on the symptom entity data and a symptom entity representation model pre-obtained by training, the symptom entity representation model including a graph convolutional neural network layer.

S303, acquiring natural text representation data corresponding to the electronic medical record and patient information representation data corresponding to the electronic medical record.

The electronic medical record includes natural text information, such as chief complaint information, current medical history information, physique examination information, and auxiliary examination information; and the electronic medical record also includes some patient information, such as age, gender, and marital history.

Specifically, the natural text information and the patient information in the electronic medical record are respectively input into a neural network pre-obtained by training, to obtain natural text representation data corresponding to the electronic medical record and patient information representation data corresponding to the electronic medical record.

Alternatively, the neural network includes, but is not limited to, a convolutional neural network, a cyclic neural network, a neural network that introduces an attention mechanism, and the like.

The convolutional neural network is took as an example, alternatively, 100 convolution kernels with length of 3, 100 convolution kernels with length of 4 and 100 convolution kernels with length of 5 are used, and dropout with a coefficient of 0.5 are adopted, and finally mean pooling for pooling processing are used, to output the natural text representation data and the patient information representation data.

S304, generating overall medical record representation data based on the symptom entity representation data, the natural text representation data, and the patient information representation data.

Specifically, the symptom entity representation data, the natural text representation data, and the patient information representation data are spliced together to obtain the overall medical record representation data.

S305, inputting the overall medical record representation data into the pre-trained classification model, and obtaining the disease prediction result corresponding to the electronic medical record data based on an output result of the classification model.

Alternatively, the classification model includes but is not limited to an MLP (multilayer perceptron) model.

As shown in FIG. 3B, FIG. 3B is a schematic diagram of a disease prediction disclosed according to Embodiment 3 of the present disclosure. Here, the reference 30 represents a process of acquiring the natural text representation data corresponding to the electronic medical record, the reference 31 represents a process of acquiring the symptom entity representation data, and the reference 32 represents a process of acquiring the patient information representation data. Specifically, the process 30 includes: extracting the natural text information from the electronic medical record, performing vector encoding on the natural text information, and then performing convolution calculation on the encoding result, and finally performing mean pooling on the convolution result to obtain the natural text representation data; the process 31 includes: extracting the symptom entity data from the electronic medical record, and perform vector encoding on the symptom entity data, then inputting the encoding result into the graph convolutional neural network layer to obtain the symptom vectorized representation data, and finally performing mean pooling on the symptom vectorized representation data to obtain the symptom entity representation data; the process 32 is similar to the process 30, including: extracting the patient information from the electronic medical record, performing vector encoding on the patient information, then performing convolution calculation on the encoding result, and finally performing mean pooling on the convolution result to obtain the patient information representation data. Based on the natural text representation data, the symptom entity representation data, and the patient information representation data, the overall medical record representation data are obtained, and disease prediction is performed based on the MLP model.

In the present embodiment, by acquiring the natural text representation data and the patient information representation data corresponding to the electronic medical record; then generating the overall medical record representation data based on the symptom entity representation data, the natural text representation data, and the patient information representation data; and finally inputting the overall medical record representation data into the classification model, and the disease prediction result is obtained. Since the overall medical record representation data include three representation data: the natural text representation data, the patient information representation data, and the symptom entity representation data, the representation data include a wide range of information and sufficient data, so that the accuracy of the finally obtained disease prediction result based on the overall medical record representation data is high.

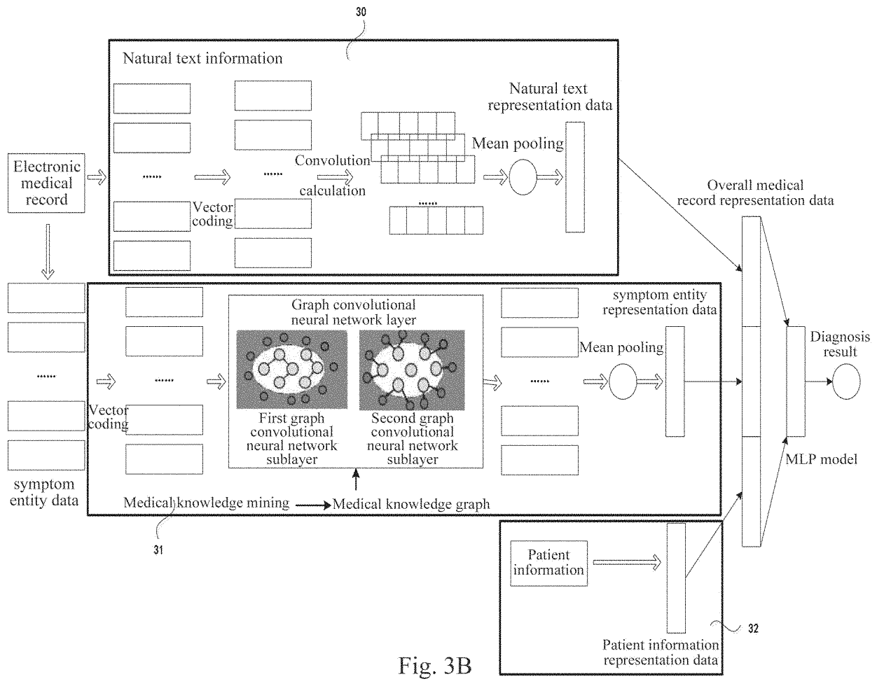

FIG. 4 is a schematic structural diagram of an apparatus for processing electronic medical record data disclosed according to Embodiment 4 of the present disclosure. The present embodiment may be applied to the situation of automatically performing disease prediction based on electronic medical record data. The apparatus in the present embodiment may be implemented by software and/or hardware, and may be integrated on any electronic device having computing capability, such as a server.

As shown in FIG. 4, an apparatus 40 for processing electronic medical record data disclosed in the present embodiment may include a symptom entity data acquisition module 41, a representation data acquisition module 42 and a disease prediction result acquisition module 43, in which:

the symptom entity data acquisition module 41, is configured to acquire symptom entity data in electronic medical record data;

the representation data acquisition module 42, is configured to obtain symptom entity representation data based on the symptom entity data and a symptom entity representation model pre-obtained by training, the symptom entity representation model comprising a graph convolutional neural network layer;

the disease prediction result acquisition module 43, is configured to obtain a disease prediction result corresponding to the electronic medical record data, based on the symptom entity representation data and a classification model pre-obtained by training.

Alternatively, the symptom entity representation model includes: a vector coding layer, the graph convolutional neural network layer, and a pooling layer;

the vector coding layer is used to encode the symptom entity data to obtain a symptom encoding vector corresponding to the symptom entity data;

the graph convolutional neural network layer is used to obtain symptom vectorized representation data fused with graph structure information based on the symptom encoding vector; and

the pooling layer is used to perform pooling processing on the symptom vectorized representation data to obtain the symptom entity representation data.

Alternatively, the apparatus further includes a medical knowledge graph construction module, configured to:

construct a medical knowledge graph, wherein the medical knowledge graph comprises at least one disease entity node and at least one symptom entity node;

there is a connection relationship between two disease entity nodes having a hyponymy relationship in the disease entity nodes; and

for any disease entity node and any symptom entity node, if a disease corresponding to the disease entity node causes a symptom corresponding to the symptom entity node to occur, then there is a connection relationship between the disease entity node and the symptom entity node; and

correspondingly, the graph convolutional neural network layer is specifically used to:

obtain the symptom vectorized representation data fused with graph structure information based on the medical knowledge graph and the symptom encoding vector.

Alternatively, the graph convolutional neural network layer includes a first graph convolutional neural network sublayer and a second graph convolutional neural network sublayer;

the first graph convolutional neural network sub-layer is used to obtain disease vectorized representation data fused with graph structure information, based on the medical knowledge graph and a disease encoding vector of a target disease entity node, the target disease entity node having a connection relationship with a target symptom entity node corresponding to the symptom entity data; and

the second graph convolutional neural network sublayer is used to obtain the symptom vectorized representation data fused with graph structure information, based on the medical knowledge graph, the symptom encoding vector, and the disease vectorized representation data.

Alternatively, the disease vectorized representation data fused with graph structure information are obtained according to the formula as follows:

$${\hat{D}}_{i} = ReLU ( {W}_{1} {D}_{i} + {\sum }_{u ∈ {N}_{p} ( i )} \frac{{W}_{2} {D}_{u}}{\vert {N}_{p} ( i ) \vert} + {\sum }_{v ∈ {N}_{c} ( i )} \frac{{W}_{3} {D}_{v}}{\vert {N}_{c} ( i ) \vert} + {B}_{1} )$$

Here, ReLU represents an activation function, W₁, W₂, W₃, and B₁ respectively represent model parameters to be trained, Nₚ(i) represents a parent node set corresponding to the target disease entity node, N_{c}(i) represents a child node set corresponding to the target disease entity node, \hat{D}ᵢ represents the disease vectorized representation data, Dᵢ represents the disease encoding vector, Dᵥ represents an encoding vector of a child node of the target disease entity node, and Dᵤ represents an encoding vector of a parent node of the target disease entity node.

Alternatively, the symptom vectorized representation data fused with graph structure information are obtained according to the formula as follows:

$${\hat{F}}_{j} = ReLU ( {W}_{4} {F}_{j} + \frac{1}{\vert {N}_{9} ( j ) \vert} {\sum }_{i ∈ {N}_{g} ( j )} {A}_{i , j} {W}_{5} {\hat{D}}_{i} + {B}_{2} )$$

ReLU represents an activation function, W₄, W₅ and B₂ respectively represent model parameters to be trained, N_{g}(j) represents a set of the target disease entity nodes, A_{i,j} represents a weight of a connection relationship between the target symptom entity node and the target disease entity node, \hat{F}ⱼ represents the symptom vectorized representation data, and Fⱼ represents the symptom encoding vector.

Alternatively, the weight A_{i,j} of the connection relationship between the target symptom entity node and the target disease entity node is determined according to the formula as follows:

$${A}_{i , j} = n {〈 {f}_{j} \vert {d}_{i} 〉}^{*} log \frac{N}{1 + n ( {d}_{i} )}$$

n [symbol] fⱼ|dᵢ [symbol] represents a frequency of the target symptom entity node presenting in the medical records with the target disease entity node as a main diagnosis, n(dᵢ) represents the total number of medical records with the target disease entity node as the main diagnosis, and N represents the total number of medical records used.

Alternatively, the disease prediction result acquisition module 43 is configured to:

acquire natural text representation data corresponding to the electronic medical record and patient information representation data corresponding to the electronic medical record;

generate overall medical record representation data based on the symptom entity representation data, the natural text representation data, and the patient information representation data; and

input the overall medical record representation data into the pre-trained classification model, and obtain the disease prediction result corresponding to the electronic medical record data based on an output result of the classification model.

The apparatus 40 for processing electronic medical record data disclosed in embodiments of the present disclosure may perform any method for processing electronic medical record data disclosed in embodiments of the present disclosure, and has the corresponding functional modules for performing the method and beneficial effects thereof. For content not described in detail in the present embodiment, reference may be made to the description in any embodiment of the method for processing electronic medical record data in the present disclosure.

According to an embodiment of the present disclosure, an electronic device and a readable storage medium are also provided.

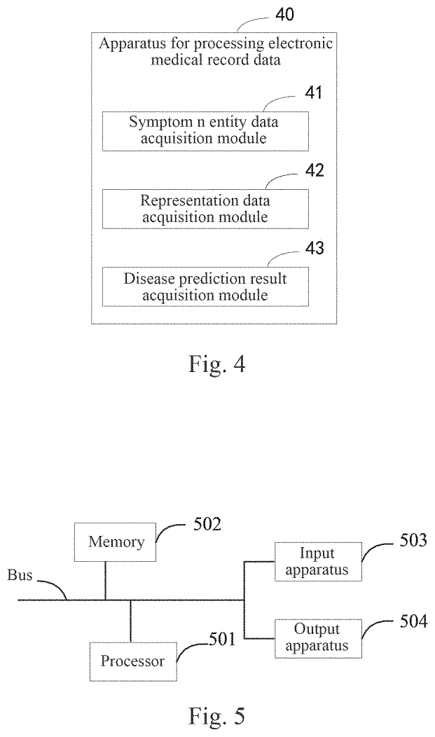

As shown in FIG. 5, which is a block diagram of an electronic device of the method for processing electronic medical record data according to an embodiment of the present disclosure. The electronic device is intended to represent various forms of digital computers, such as laptop computers, desktop computers, workbenches, personal digital assistants, servers, blade servers, mainframe computers, and other suitable computers. The electronic device may also represent various forms of mobile apparatuses, such as personal digital processors, cellular phones, smart phones, wearable devices, and other similar computing apparatuses. The components shown herein, their connections and relationships, and their functions are merely examples, and are not intended to limit the implementation of the present disclosure described and/or claimed herein.

As shown in FIG. 5, the electronic device includes: one or more processors 501, a memory 502, and interfaces for connecting various components, including high-speed interfaces and low-speed interfaces. The various components are connected to each other using different buses, and may be installed on a common motherboard or in other methods as needed. The processor may process instructions executed within the electronic device, including instructions stored in or on the memory to display graphic information of GUI on an external input/output apparatus (such as a display device coupled to an interface). In other embodiments, a plurality of processors and/or a plurality of buses may be used together with a plurality of memories and a plurality of memories if desired. Similarly, a plurality of electronic devices may be connected, and the devices provide some necessary operations, for example, as a server array, a set of blade servers, or a multi-processor system. In FIG. 5, one processor 501 is used as an example.

The memory 502 is a non-transitory computer readable storage medium provided by some embodiments of the present disclosure. The memory stores instructions executable by at least one processor, so that the at least one processor performs the method for processing electronic medical record data provided by embodiments of the present disclosure. The non-transitory computer readable storage medium of some embodiments of the present disclosure stores computer instructions for causing a computer to perform the method for processing electronic medical record data provided by the present disclosure.

The memory 502, as a non-transitory computer readable storage medium, may be used to store non-transitory software programs, non-transitory computer executable programs and modules, such as program instructions/modules corresponding to the method for processing electronic medical record data in the embodiments of the present disclosure (for example, the symptom entity data acquisition module 41, the representation data acquisition module 42 and the disease prediction result acquisition module 43 as shown in FIG. 4). The processor 501 executes the non-transitory software programs, instructions, and modules stored in the memory 502 to execute various functional applications and data processing of the server, that is, to implement the method for processing electronic medical record data in the foregoing method embodiments.

The memory 502 may include a storage program area and a storage data area, where the storage program area may store an operating system and at least one function required application program; and the storage data area may store data created by the use of the electronic device according to the method for processing electronic medical record data, etc. In addition, the memory 502 may include a high-speed random access memory, and may also include a non-transitory memory, such as at least one magnetic disk storage device, a flash memory device, or other non-transitory solid-state storage devices. In some embodiments, the memory 502 may optionally include memories remotely provided with respect to the processor 501, and these remote memories may be connected to the electronic device of the method for processing electronic medical record data through a network. Examples of the above network include but are not limited to the Internet, intranet, local area network, mobile communication network, and combinations thereof.

The electronic device of the method for processing electronic medical record data may also include: an input apparatus 503 and an output apparatus 504. The processor 501, the memory 502, the input apparatus 503, and the output apparatus 504 may be connected through a bus or in other methods. In FIG. 5, connection through a bus is used as an example.

The input apparatus 503 may receive input digital or character information, and generate key signal inputs related to user settings and function control of the electronic device of the method for processing electronic medical record data, such as touch screen, keypad, mouse, trackpad, touchpad, pointing stick, one or more mouse buttons, trackball, joystick and other input apparatuses. The output apparatus 504 may include a display device, an auxiliary lighting apparatus (for example, LED), a tactile feedback apparatus (for example, a vibration motor), and the like. The display device may include, but is not limited to, a liquid crystal display (LCD), a light emitting diode (LED) display, and a plasma display. In some embodiments, the display device may be a touch screen.

Various embodiments of the systems and technologies described herein may be implemented in digital electronic circuit systems, integrated circuit systems, dedicated ASICs (application specific integrated circuits), computer hardware, firmware, software, and/or combinations thereof. These various embodiments may include: being implemented in one or more computer programs that can be executed and/or interpreted on a programmable system that includes at least one programmable processor. The programmable processor may be a dedicated or general-purpose programmable processor, and may receive data and instructions from a storage system, at least one input apparatus, and at least one output apparatus, and transmit the data and instructions to the storage system, the at least one input apparatus, and the at least one output apparatus.

These computing programs (also referred to as programs, software, software applications, or codes) include machine instructions of the programmable processor and may use high-level processes and/or object-oriented programming languages, and/or assembly/machine languages to implement these computing programs. As used herein, the terms “machine readable medium” and “computer readable medium” refer to any computer program product, device, and/or apparatus (for example, magnetic disk, optical disk, memory, programmable logic apparatus (PLD)) used to provide machine instructions and/or data to the programmable processor, including machine readable medium that receives machine instructions as machine readable signals. The term “machine readable signal” refers to any signal used to provide machine instructions and/or data to the programmable processor.

In order to provide interaction with a user, the systems and technologies described herein may be implemented on a computer, the computer has: a display apparatus for displaying information to the user (for example, CRT (cathode ray tube) or LCD (liquid crystal display) monitor); and a keyboard and a pointing apparatus (for example, mouse or trackball), and the user may use the keyboard and the pointing apparatus to provide input to the computer. Other types of apparatuses may also be used to provide interaction with the user; for example, feedback provided to the user may be any form of sensory feedback (for example, visual feedback, auditory feedback, or tactile feedback); and any form (including acoustic input, voice input, or tactile input) may be used to receive input from the user.

The systems and technologies described herein may be implemented in a computing system that includes backend components (e.g., as a data server), or a computing system that includes middleware components (e.g., application server), or a computing system that includes frontend components (for example, a user computer having a graphical user interface or a web browser, through which the user may interact with the implementations of the systems and the technologies described herein), or a computing system that includes any combination of such backend components, middleware components, or frontend components. The components of the system may be interconnected by any form or medium of digital data communication (e.g., communication network). Examples of the communication network include: local area networks (LAN), wide area networks (WAN), the Internet, and blockchain networks.

The computer system may include a client and a server. The client and the server are generally far from each other and usually interact through the communication network. The relationship between the client and the server is generated by computer programs that run on the corresponding computer and have a client-server relationship with each other.

According to the technical solution of embodiments of the present disclosure, the symptom entity representation data are acquired based on the acquired symptom entity data and the symptom entity representation model pre-obtained by training, where the symptom entity representation model includes a graph convolutional neural network layer, and then the disease prediction result corresponding to the electronic medical record data is obtained based on the symptom entity representation data and the classification model pre-obtained by training. Since the symptom entity representation model pre-obtained by training includes the graph convolutional neural network layer, the output symptom entity representation data have high accuracy, so that the accuracy of the finally obtained disease prediction result corresponding to the electronic medical record data is also high.

It should be understood that the various forms of processes shown above may be used to reorder, add, or delete steps. For example, the steps described in some embodiments of the present disclosure may be performed in parallel, sequentially, or in different orders. As long as the desired results of the technical solution disclosed in some embodiments of the present disclosure can be achieved, no limitation is made herein.

The above specific embodiments do not constitute limitation on the protection scope of the present disclosure. Those skilled in the art should understand that various modifications, combinations, sub-combinations and substitutions may be made according to design requirements and other factors. Any modification, equivalent replacement and improvement made within the spirit and principle of the present disclosure shall be included in the protection scope of the present disclosure.

What is claimed is:

1. A method for processing electronic medical record data, the method comprising:
acquiring symptom entity data in the electronic medical record data;
obtaining symptom entity representation data based on the symptom entity data and a symptom entity representation model pre-obtained by training, the symptom entity representation model comprising a graph convolutional neural network layer; and
obtaining a disease prediction result corresponding to the electronic medical record data, based on the symptom entity representation data and a classification model pre-obtained by training.

2. The method according to claim 1, wherein the symptom entity representation model comprises: a vector coding layer, the graph convolutional neural network layer, and a pooling layer;
the vector coding layer is used to encode the symptom entity data to obtain a symptom encoding vector corresponding to the symptom entity data;
the graph convolutional neural network layer is used to obtain symptom vectorized representation data fused with graph structure information based on the symptom encoding vector; and
the pooling layer is used to perform pooling processing on the symptom vectorized representation data to obtain the symptom entity representation data.

3. The method according to claim 2, wherein, before the acquiring the symptom entity data in electronic medical record data, the method further comprises:
constructing a medical knowledge graph, wherein the medical knowledge graph comprises at least one disease entity node and at least one symptom entity node;
there is a connection relationship between two disease entity nodes having a hyponymy relationship in the disease entity nodes;
for any disease entity node and any symptom entity node, if a disease corresponding to the disease entity node causes a symptom corresponding to the symptom entity node to occur, then there is a connection relationship between the disease entity node and the symptom entity node; and
correspondingly, the graph convolutional neural network layer is used to:
obtain the symptom vectorized representation data fused with graph structure information based on the medical knowledge graph and the symptom encoding vector.

4. The method according to claim 3, wherein the graph convolutional neural network layer comprises a first graph convolutional neural network sublayer and a second graph convolutional neural network sublayer;
the first graph convolutional neural network sub-layer is used to obtain disease vectorized representation data fused with graph structure information, based on the medical knowledge graph and a disease encoding vector of a target disease entity node, the target disease entity node having a connection relationship with a target symptom entity node corresponding to the symptom entity data; and
the second graph convolutional neural network sublayer is used to obtain the symptom vectorized representation data fused with graph structure information, based on the medical knowledge graph, the symptom encoding vector, and the disease vectorized representation data.

5. The method according to claim 4, wherein, the disease vectorized representation data fused with graph structure information are obtained according to a formula as follows:
$${\hat{D}}_{i} = ReLU ( {W}_{1} {D}_{i} + {\sum }_{u ∈ {N}_{p} ( i )} \frac{{W}_{2} {D}_{u}}{\vert {N}_{p} ( i ) \vert} + {\sum }_{v ∈ {N}_{c} ( i )} \frac{{W}_{3} {D}_{v}}{\vert {N}_{c} ( i ) \vert} + {B}_{1} )$$
wherein, ReLU represents an activation function, W₁, W₂, W₃, and B₁ respectively represent model parameters to be trained, Nₚ(i) represents a parent node set corresponding to the target disease entity node, N_{c}(i) represents a child node set corresponding to the target disease entity node, \hat{D}ᵢ represents the disease vectorized representation data, Dᵢ represents the disease encoding vector, Dᵥ represents an encoding vector of a child node of the target disease entity node, and Dᵤ represents an encoding vector of a parent node of the target disease entity node.

6. The method according to claim 4, wherein, the symptom vectorized representation data fused with graph structure information are obtained according to a formula as follows:
$${\hat{F}}_{j} = ReLU ( {W}_{4} {F}_{j} + \frac{1}{\vert {N}_{g} ( j ) \vert} {\sum }_{i ∈ {N}_{g} ( j )} {A}_{i , j} {W}_{5} {\hat{D}}_{i} + {B}_{2} )$$
wherein, ReLU represents an activation function, W₄, W₅ and B₂ respectively represent model parameters to be trained, N_{g}(j) represents a set of the target disease entity nodes, A_{i,j} represents a weight of a connection relationship between the target symptom entity node and the target disease entity node, \hat{F}ⱼ represents the symptom vectorized representation data, and Fⱼ represents the symptom encoding vector.

7. The method according to claim 6, wherein, the weight A_{i,j} of the connection relationship between the target symptom entity node and the target disease entity node is determined according to a formula as follows:
$${A}_{i , j} = n {〈 {f}_{j} \vert {d}_{i} 〉}^{*} log \frac{N}{1 + n ( {d}_{i} )}$$
wherein, n [symbol] fⱼ|dᵢ [symbol] represents a frequency of the target symptom entity node presenting in medical records with the target disease entity node as a main diagnosis, n(dᵢ) represents a total number of the medical records with the target disease entity node as the main diagnosis, and N represents a total number of medical records used.

8. The method according to claim 1, wherein, the obtaining the disease prediction result corresponding to the electronic medical record data, based on the symptom entity representation data and the classification model obtained by pre-training, comprises:
acquiring natural text representation data corresponding to the electronic medical record and patient information representation data corresponding to the electronic medical record;
generating overall medical record representation data based on the symptom entity representation data, the natural text representation data, and the patient information representation data; and
inputting the overall medical record representation data into the pre-trained classification model, and obtaining the disease prediction result corresponding to the electronic medical record data based on an output result of the classification model.

9. An electronic device, comprising:
at least one processor; and
a memory, communicatively connected to the at least one processor; wherein,
the memory, stores instructions executable by the at least one processor, the instructions, when executed by the at least one processor, cause the at least one processor to perform operations comprising:
acquiring symptom entity data in an electronic medical record data;
obtaining symptom entity representation data based on the symptom entity data and a symptom entity representation model pre-obtained by training, the symptom entity representation model comprising a graph convolutional neural network layer; and
obtaining a disease prediction result corresponding to the electronic medical record data, based on the symptom entity representation data and a classification model pre-obtained by training.

10. The device according to claim 9, wherein the symptom entity representation model comprises: a vector coding layer, the graph convolutional neural network layer, and a pooling layer;
the vector coding layer is used to encode the symptom entity data to obtain a symptom encoding vector corresponding to the symptom entity data;
the graph convolutional neural network layer is used to obtain symptom vectorized representation data fused with graph structure information based on the symptom encoding vector; and
the pooling layer is used to perform pooling processing on the symptom vectorized representation data to obtain the symptom entity representation data.

11. The device according to claim 10, wherein, before the acquiring the symptom entity data in electronic medical record data, the operations further comprise:
constructing a medical knowledge graph, wherein the medical knowledge graph comprises at least one disease entity node and at least one symptom entity node;
there is a connection relationship between two disease entity nodes having a hyponymy relationship in the disease entity nodes;
for any disease entity node and any symptom entity node, if a disease corresponding to the disease entity node causes a symptom corresponding to the symptom entity node to occur, then there is a connection relationship between the disease entity node and the symptom entity node; and
correspondingly, the graph convolutional neural network layer is used to:
obtain the symptom vectorized representation data fused with graph structure information based on the medical knowledge graph and the symptom encoding vector.

12. The device according to claim 11, wherein the graph convolutional neural network layer comprises a first graph convolutional neural network sublayer and a second graph convolutional neural network sublayer;
the first graph convolutional neural network sub-layer is used to obtain disease vectorized representation data fused with graph structure information, based on the medical knowledge graph and a disease encoding vector of a target disease entity node, the target disease entity node having a connection relationship with a target symptom entity node corresponding to the symptom entity data; and
the second graph convolutional neural network sublayer is used to obtain the symptom vectorized representation data fused with graph structure information, based on the medical knowledge graph, the symptom encoding vector, and the disease vectorized representation data.

13. The device according to claim 12, wherein, the disease vectorized representation data fused with graph structure information are obtained according to a formula as follows:
$${\hat{D}}_{i} = ReLU ( {W}_{1} {D}_{i} + {\sum }_{u ∈ {N}_{p} ( i )} \frac{{W}_{2} {D}_{u}}{\vert {N}_{p} ( i ) \vert} + {\sum }_{v ∈ {N}_{c} ( i )} \frac{{W}_{3} {D}_{v}}{\vert {N}_{c} ( i ) \vert} + {B}_{1} )$$
wherein, ReLU represents an activation function, W₁, W₂, W₃, and B₁ respectively represent model parameters to be trained, Nₚ(i) represents a parent node set corresponding to the target disease entity node, N_{c}(i) represents a child node set corresponding to the target disease entity node, \hat{D}ᵢ represents the disease vectorized representation data, Dᵢ represents the disease encoding vector, Dᵥ represents an encoding vector of a child node of the target disease entity node, and Dᵤ represents an encoding vector of a parent node of the target disease entity node.

14. The device according to claim 12, wherein, the symptom vectorized representation data fused with graph structure information are obtained according to a formula as follows:
$${\hat{F}}_{j} = ReLU ( {W}_{4} {F}_{j} + \frac{1}{\vert {N}_{g} ( j ) \vert} {\sum }_{i ∈ {N}_{g} ( j )} {A}_{i , j} {W}_{5} {\hat{D}}_{i} + {B}_{2} )$$
wherein, ReLU represents an activation function, W₄, W₅ and B₂ respectively represent model parameters to be trained, N_{g}(j) represents a set of the target disease entity nodes, A_{i,j} represents a weight of a connection relationship between the target symptom entity node and the target disease entity node, represents the symptom vectorized representation data, and Fⱼ represents the symptom encoding vector.

15. The device according to claim 14, wherein, the weight of the connection relationship between the target symptom entity node and the target disease entity node is determined according to a formula as follows:
$${A}_{i , j} = n {〈 {f}_{j} \vert {d}_{i} 〉}^{*} log \frac{N}{1 + n ( {d}_{i} )}$$
wherein, n [symbol] fⱼ|dᵢ [symbol] represents a frequency of the target symptom entity node presenting in medical records with the target disease entity node as a main diagnosis, n(dᵢ) represents a total number of the medical records with the target disease entity node as the main diagnosis, and N represents a total number of medical records used.

16. The device according to claim 9, wherein, the obtaining the disease prediction result corresponding to the electronic medical record data, based on the symptom entity representation data and the classification model obtained by pre-training, comprises:
acquiring natural text representation data corresponding to the electronic medical record and patient information representation data corresponding to the electronic medical record;
generating overall medical record representation data based on the symptom entity representation data, the natural text representation data, and the patient information representation data; and
inputting the overall medical record representation data into the pre-trained classification model, and obtaining the disease prediction result corresponding to the electronic medical record data based on an output result of the classification model.

17. A non-transitory computer readable storage medium, storing computer instructions, the computer instructions, when executed by a computer, cause the computer to perform operations comprising:
acquiring symptom entity data in an electronic medical record data;
obtaining symptom entity representation data based on the symptom entity data and a symptom entity representation model pre-obtained by training, the symptom entity representation model comprising a graph convolutional neural network layer; and
obtaining a disease prediction result corresponding to the electronic medical record data, based on the symptom entity representation data and a classification model pre-obtained by training.
